(19) United States
(12) Patent Application Publication
(10) Pub. No.: US 2011/0313392 A1
(43) Pub. Date: Dec. 22, 2011

(54) STEERABLE EPIDURAL CATHETER

(75) Inventors: Ebby Varghese, Columbia, MO (US); Zachary Hafez, Columbia, MO (US); Nicole Patino, Columbia, MO (US)

(73) Assignee: THE CURATORS OF THE UNIVERSITY OF MISSOURI, Columbia, MO (US)

(21) Appl. No.: 13/164,517

(22) Filed: Jun. 20, 2011

Related U.S. Application Data
(60) Provisional application No. 61/397,943, filed on Jun. 18, 2010.
(60) Provisional application No. 61/455,199, filed on Oct. 15, 2010.

(51) Int. Cl. A61M 25/092; B23P 17/04

(52) U.S. Cl. 604/500; 604/95.04; 29/428

(57) ABSTRACT

A steerable catheter device which can be steered to locations within the epidural space, as well as methods of use and manufacture are disclosed. The steerable catheter provides a site-specific drug delivery system for accessing the epidural space. The catheter devices includes a steerable wire or a liner extending from a proximal end of the catheter to the catheter's distal end. Forces applied to the wire or the liner cause the distal end to deflect and/or rotate to reach a targeted epidural site.

### CROSS-REFERENCE TO RELATED APPLICATIONS

This is a non-provisional and claims benefit to U.S. Provisional Application No. 61/397,943 entitled “Steerable Catheter Device and Uses Thereof” filed on Jun. 18, 2010, and U.S. Provisional Application No. 61/455,199 entitled “Steerable Epidural Catheter” filed on Oct. 15, 2010, both of which are hereby incorporated by reference.

### FIELD

The present application relates to surgical devices and more particularly to a steerable catheter that can be guided to site-specific locations.

### BACKGROUND

The most common interventional pain management procedures are used to manage chronic neck and/or back pain, as well as pain associated with labor and delivery. These procedures typically involve epidural injections to deliver pain reducing medicines to the epidural space. Epidural injections may be made using a direct needle injection at the site of pain (transforaminal approach) or using a catheter maneuvered into the epidural space (interlaminar approach). Although, the transforaminal approach may be more diagnostic and provide greater long term pain management than the interlaminar approach, it carries an increased risk of complications, such as inadvertent vascular or nerve injury, spinal cord injury and death.

Guided catheters have been employed to manage pain using the interlaminar approach. These types of catheters are typically designed for intravascular areas and are not optimized for epidural procedures. Vascular-type guided catheters often rely on the use of a guide wire to position the catheter within the vascular system. After positioning the guide wire near the treatment site, a separate, hollow catheter may then be threaded over the guide wire and maneuvered to the desired location. The guide wire is then withdrawn and the catheter left in place. After the procedure is completed, the guide wire must be re-inserted into the inner lumen of the catheter and the catheter and guide wire collectively withdrawn. However, this process can be cumbersome, time-consuming, and potentially increase the risk of injury to the patient.

### SUMMARY

In one embodiment, a steerable catheter device may include an elongated catheter body defining a proximal portion and a distal portion and a lumen formed along the elongated catheter body. The lumen defines a proximal end and a distal end with a reinforcing coil disposed within the lumen for supporting the elongated catheter body. A steerable wire extends proximate or within the lumen from the proximal end of the lumen to a distal tip defined at the distal end of the elongated catheter body, wherein the steerable wire is affixed to the distal tip. Actuation of the steerable wire at the proximal end of the elongated catheter body causes at least one of a rotation and a deflection of the distal tip.

In another embodiment, a steerable catheter device may include an elongated catheter body defining a proximal portion and a distal portion and a lumen formed along the elongated catheter body. The lumen defines a proximal end and a distal end with a reinforcing coil disposed within the lumen for supporting the elongated catheter body. A steerable liner extends proximate or within the lumen from the proximal end of the lumen to a distal tip defined at the distal end of the elongated catheter body, wherein the steerable liner is affixed to the distal tip. Actuation of the steerable liner at the proximal end of the elongated catheter body causes at least one of a rotation and a deflection of the distal tip.

In yet another embodiment, a method for delivering a drug to an epidural space may include:
accessing an area adjacent to the epidural space using a steerable catheter comprising:
   an elongated catheter body defining a proximal portion and a distal portion and a lumen along the elongated catheter body, the lumen having a proximal end and a distal end with a reinforcing coil disposed within the lumen for supporting the elongated catheter body, the lumen containing a steerable wire extending proximate or within the lumen from the proximal end of the lumen to a distal tip defined at the distal end of the elongated catheter body, wherein the steerable wire is affixed to the distal tip, wherein actuation of the steerable wire at the proximal end of the elongated catheter body causes at least one of a rotation and a deflection of the distal tip;
applying a force to the steering mechanism to at least one of a deflection and a rotation of the distal end into the epidural space;
providing a liquid through the lumen of the catheter;
transmitting the liquid from the proximal end of the lumen to the distal end of the lumen positioned within the epidural space; and
removing the elongated catheter body.

In an embodiment, a method for manufacturing a steerable catheter device may include:
forming an elongated catheter body defining a distal portion and a proximal portion, the elongated catheter body defining a lumen having a proximal end and a distal end, wherein the distal portion of the elongated catheter body defining a distal tip; and
inserting a steerable wire along the lumen from the proximal portion of the elongated catheter body to the distal tip, wherein actuating the steerable wire causes at least one of a rotation and a deflection of the distal tip; and
affixing an end of the steerable wire to the distal tip.

In another embodiment, a method for manufacturing a steerable catheter device may include:
forming an elongated catheter body defining a distal portion and a proximal portion, the elongated catheter body defining a lumen having a proximal end and a distal end, wherein the distal portion of the elongated body defining a distal tip; and
inserting a steerable wire along the lumen from the proximal portion of the elongated catheter body to the distal tip, wherein actuating the steerable wire causes at least one of a rotation and a deflection of the distal tip; and
affixing an end of the steerable line to the distal tip.

Additional objectives, advantages, and novel features will be set forth in the description which follows or will become apparent to those skilled in the art upon examination of the drawings and detailed description which follows.

### BRIEF DESCRIPTION OF THE DRAWINGS

FIG. 1 is an illustration of a prior art epidural site treatment system;

FIG. 2 is a partial cross-sectional side view showing one embodiment of a steerable catheter;

FIG. 3 is a side view illustrating the distal end of the steerable catheter;

FIGS. 4A and 4B are side views the deflection action of the steerable catheter;

FIG. 5 is a side view showing the collar of the steerable catheter;

FIG. 6 is a side view of another embodiment of the steerable catheter engaged to a steering mechanism;

FIG. 7 is a view of a steering mechanism of the steerable catheter; and

FIG. 8 is a cross-sectional view of tension device of the steerable catheter.

Corresponding reference characters indicate corresponding elements among the view of the drawings. The headings used in the figures should not be interpreted to limit the scope of the claims.

### DETAILED DESCRIPTION

Aspects of the present disclosure include devices and methods for a steerable catheter. In particular, the steerable catheter includes a steerable wire or a bent liner incorporated proximate or within the lumen of the steerable catheter to maneuver the steerable catheter by deflection and/or rotation of the distal tip defined at a distal end of the steerable catheter. The steerable catheter functions without a removable guide wire for positioning and removing the steerable catheter

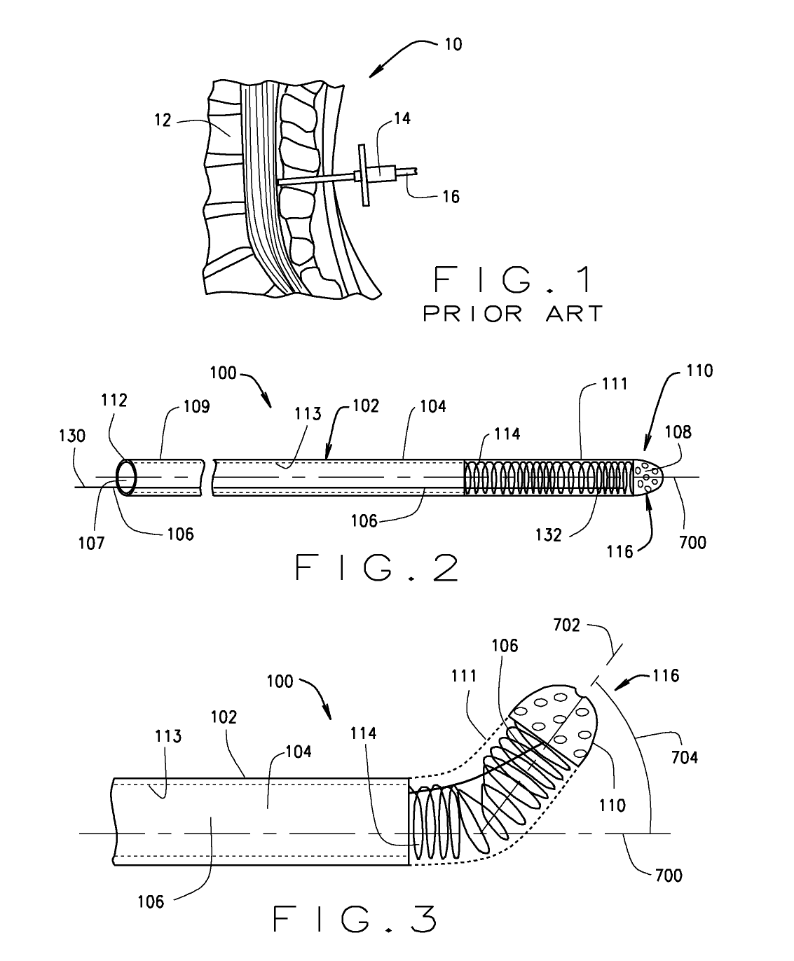

Referring to the drawings, a prior art system 10 to access an epidural site 12 of a mammalian body is illustrated and generally indicated as 10 in FIG. 1. The prior art system 10 includes an epidural needle 14 that is used to puncture the skin and access the epidural treatment site 12. A guide wire (not shown) and a catheter 16 are collectively inserted through the lumen of the needle 14 which deploys the catheter 16 at the treatment site 12. However, the use of the guide wire to maneuver the catheter 16 through the needle 14 can increase the risk of injury to surrounding tissue and may also require additional equipment to properly manipulate the guide wire and catheter 16.

Referring to FIG. 2, one embodiment of a steerable catheter generally indicated as 100 is illustrated which overcomes the issues related to the prior art system 10. The steerable catheter 100 includes an elongated catheter body 102 having a proximal portion 109 defining an opening 107 and a distal portion 111 defining a distal tip 108. In addition, the opening 107 is in fluid flow communication with a lumen 104 (shown in phantom lines) that extends longitudinally along the elongated catheter body 102. In addition, the lumen 104 has a proximal end 112 in communication with the opening 107 and a distal end 110 that communicates with the distal tip 108. The elongated catheter body 102 may have a diameter between in 1 mm and 5 mm. In one embodiment, the elongated body 102 has a diameter equal to or less than 1.5 mm.

A steerable wire 106 may be disposed within the lumen 104 having a proximal end 130 that extends outwardly from the opening 107 and a distal end 132 that is positioned proximate or within the distal tip 108. In one embodiment, the steerable wire 106 may be manipulated to deflect and/or rotate the distal tip 108 of the elongated catheter body 102 as shall be discussed in greater detail below. In other embodiments, the steerable wire 106 may be made from a malleable metal material, a bendable plastic material, or a metal material having memory retention characteristics. As shown, the steerable wire 106 may extend from the distal end 110 to a proximal end 112 of the elongated catheter body 102 in parallel orientation relative to the longitudinal axis 700 of the elongated catheter body 102. The lumen 104 also provides fluid flow communication between the proximal end 112 and distal end 110 of the elongated catheter body 102 such that a medicinal or therapeutic agent, for example a pharmaceutical drug, may be injected at the proximal end 112 and then discharged at the distal end 110 of the lumen 104. In some embodiments, a reinforced coil 114 may be disposed along a portion or entire length of the lumen 104 to provide a reinforcing structure to the elongated catheter body 102. In one embodiment, the steerable wire 106 may be disposed longitudinally through the reinforcing coil 114; however, in other embodiments, the steerable wire 106 may be disposed freely within the lumen 104, attached along the inner surface 113 of the lumen 104 or embedded under the inner surface 113 within the elongated catheter body 102. The reinforcing coil 114 also serves to structurally connect the distal tip 108 with distal portion 132 of the elongated catheter body 102. In one embodiment, the reinforcing coil 114 may be a variable tension coiled spring. In other embodiments, the reinforcing coil 114 may be a woven, helical, or braided reinforcement structure within the elongated catheter body 102.

As further shown, the distal tip 108 includes a plurality of holes 116 that are in fluid flow communication with the lumen 104 such that a liquid, for example a therapeutic agent, injected through the opening 107 of the lumen 104 may pass through the plurality of holes 116 and into the epidural space or other body cavity. The plurality of holes 116 also reduces the probability of any debris or obstruction from completely preventing fluid flow communication through the plurality of holes 116. In some embodiments, the distal tip 108 may be rounded and have a generally hemispherical configuration. This generally hemispherical configuration of the distal tip 108 also minimizes injuries to the tissue at and around the epidural site 12.

As noted above, the proximal end 130 of the steerable wire 106 may be manually manipulated to cause the distal tip 108 to rotate and/or deflect away relative to a longitudinal axis 700 of the elongated catheter body 102. In particular, distal tip 108 may be deflected such that a center axis 702 of the distal tip 108 is deflected at an angle 704 relative to the longitudinal axis 700. In some embodiments, the angle 704 of deflection may be between zero to ninety degrees, although in some embodiments the angle 704 of deflection may be between twenty to forty five degrees. The manipulation of the steerable wire 106 may include the application of a tensile force, a compressive force, a rotational torque, or a force perpendicular applied along the longitudinal axis 700.

In various aspects, a combination of manipulative forces may be used to manipulate the steerable wire 106. For example, a first force may deflect the center axis 702 of distal tip 108 from the longitudinal axis 700 at a deflection angle 704 and a second force, such as a rotational force may be applied to rotate the distal tip 108 about the center axis 702. In some embodiments, the first and second forces may be applied sequentially or concurrently to deflect and rotate the distal tip 108.

Referring to FIG. 3, the steerable catheter 100 may include a reinforcing coil 114 defining a structural defect 118. For example, the structural defect 118 may be generally a wedge-shaped formation defined by one or more loops of the reinforcing coil 114, which may be formed by the intentional strengthening of one or more loops of the reinforcing coil, or the fusion of two or more loops of the reinforcing coil 114. The structural defect 118 facilitates the deflection and/or rotation of the distal tip 108 when the steerable wire 106 is manipulated. In one aspect, the structural defect 118 reduces the strength of the reinforcing coil 114 at the formation of the structural defect 118 along the reinforcing coil 114, and therefore makes the elongated catheter body 102 more flexible at the distal portion 111 and susceptible to the rotation and/or deflection caused by manipulation of the steerable wire 206. Although the structural defect 118 may be wedge-shaped, the structural defect 118 may have other shapes, such as a symmetrical shape, an asymmetrical shape, a rectangular shape, a square shape or combination of above shapes.

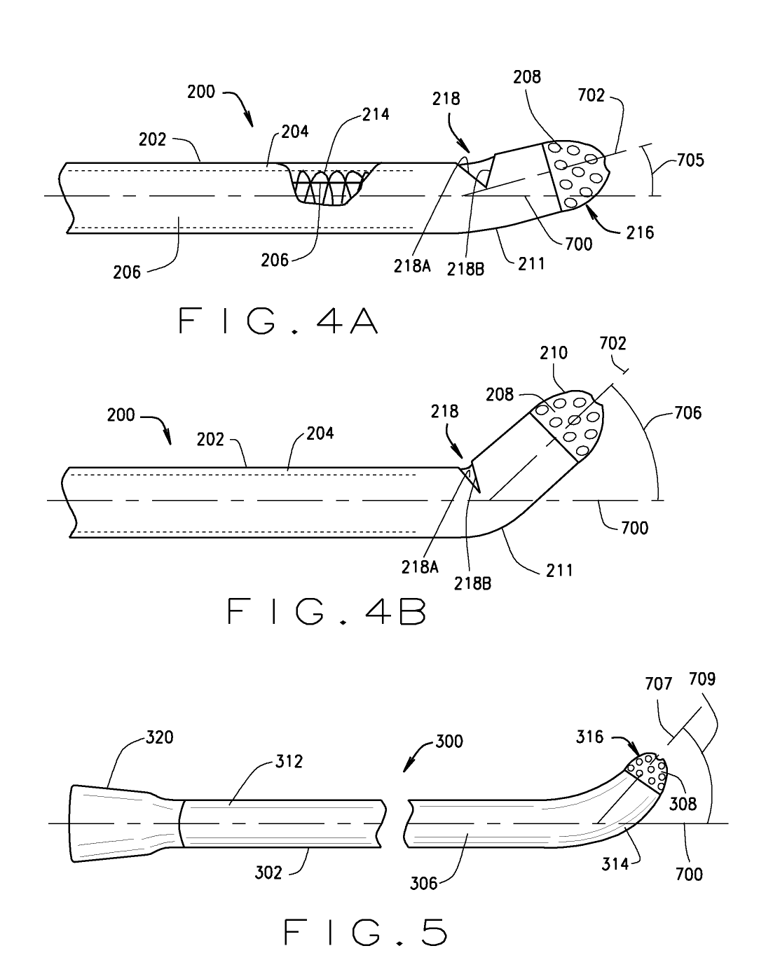

Referring to FIGS. 4A and 4B, the steerable catheter, designated 200, has substantially the same configuration as the steerable catheter 100 having an elongated catheter body 202 that defines a lumen 204 having a reinforcing coil 214 and a steerable wire 206 disposed longitudinally proximate or within the lumen 204. The lumen 204 is in fluid flow communication with a plurality of holes 208 and extends substantially along longitudinal axis 700 when the distal portion 211 is in a straight configuration. In this particular embodiment, the elongated catheter body 202 defines a structural defect 218 for facilitating the deflection and/or rotation of the distal tip 208 rather than the reinforcing coil 214 having the structural defect. In some embodiments, the structural defect 218 may have a wedge-shape configuration defined along the elongated catheter body 202, one or more pleats incorporated into the elongated catheter body, or the structural defect 218 may constitute the partial removal of a localized portion of the external or internal surface of the elongated catheter body 202. In one aspect, the structural defect 218 reduces the strength of the elongated catheter body 302 at the location of the structural defect 218, and therefore makes the distal portion 211 of the elongated catheter body 202 more flexible and susceptible to the rotation and/or deflection caused by manipulation of the steerable wire 206. As shown in FIG. 4A, manipulation of the steerable wire 206 causes center axis 702 of the distal tip 208 to deflect at an angle 705 relative to the longitudinal axis 700, thereby bringing the opposing sides 218A and 218B of the structural defect 218 toward each other. Referring to FIG. 4B, further manipulation of the steerable wire 206 causes an increase in the deflection angle 706 between the center axis 702 and the longitudinal axis 700 and brings the opposing sides 218A and 218B of the structural defect 218 closer together to facilitate the deflection of the distal tip 208.

Referring now to FIG. 5, another embodiment of the steerable catheter, designated 300, is illustrated. In this embodiment, the steerable catheter 300 includes an elongated catheter body 302 defining a lumen 304 configured to receive a steerable liner 306 therein. The steerable liner 306 defines a proximal portion 312 and a distal portion 314 that extends substantially the length of the elongated catheter body 302 such that manipulation of the proximal portion 312 of the steerable liner 306 causes the distal portion 314 to deflect and/or rotate the distal tip 308. The steerable liner 306 is a substantially rigid tubular structure, such as a hollow stylette, having a diameter less than the diameter of the lumen 304. The steerable liner 306 has a generally angled configuration, where the distal portion 314 of the steerable liner 306 may be angled relative to the proximal end 312 of the steerable liner 306. In some embodiments, the distal portion 314 of the steerable liner 306 may be angled in a range between zero degrees to ninety degrees relative to the longitudinal axis 700. In one aspect, the steerable liner 306 is threaded through the lumen 304 of the elongated catheter body 302, such that the center axis 707 of distal tip 308 of the elongated catheter body 302 is deflected from the longitudinal axis 700 by an angle 709 that is substantially equal to the deflection of the distal portion 314 of the steerable liner 306. In some embodiments, the steerable catheter 300 may also include a steering collar 320 engaged to the proximal portion 312 of the steerable liner 302 to articulate the distal portion 314 of the steerable liner 306. In one embodiment, the steering collar 320 is mechanically engaged with the steerable liner 306 to impart a force upon the steerable liner 306. The steerable liner 306 may be subjected to tensile force, a compressive force, a rotational torque, or a perpendicular force along the longitudinal axis 700 of the steerable catheter 300.

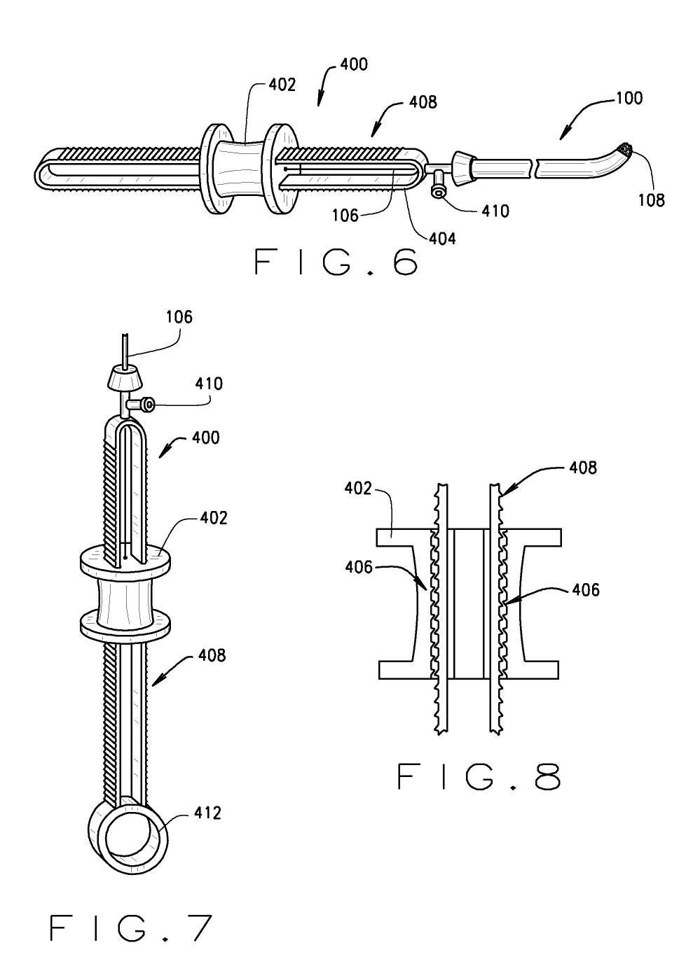

Referring now to FIGS. 6-8, the steerable catheter 100 may be operatively engaged to a steering mechanism, generally indicated as 400, for articulating the steering wire 106. In particular, operation of the steering mechanism 400 imparts a force that manipulates the steerable wire 106 and causes the deflection and/or rotation of the distal tip 108 as described above. The steering mechanism 400 may include a tension device 402 and a rotational steering device 404. The tension device 402 may be used to impart and maintain constant tensile and compressive forces on the steerable wire 106, while the rotational steering device 404 is used to impart a rotational force on the steerable wire 106. In one aspect, the functionality of the tension device 402 and a rotational steering device 404 may be incorporated into single steering device that manipulates the steerable wire 106.

As shown in FIG. 8, the tension device 402 may also incorporate locking features 406 to maintain a constant force to the steerable wire 106. Referring to FIGS. 7 and 8, the steering mechanism 400 may include a plurality of ratchet teeth 408 that are mechanically engaged to locking features 406 of the tension device 402 to prevent undesired motion of the tension device. For example, the locking features 406 may be a series of pawls or other projections to slidably engage with the plurality of ratchet teeth 408 and prevent undesired retrograde motion of the tension device 402.

In various other embodiments, the steering mechanism 400 may also include an injection port, 410 and a handle 412. The injection port 410 may used to deliver medications to the epidural site 12 through the lumen 104 of the elongated catheter body 102, while the handle 412 provides a means to operate the steering mechanism 400. The steering mechanism 400 may include a plurality of ratchet teeth 404 that are mechanically engaged by the tension device 402 to prevent undesired motion of the steering mechanism 400 and the steerable wire 106. In another embodiment, the steering mechanism 400 may mechanically engaged to the steerable catheter 100. The steering mechanism 400 may be used to impart and maintain constant tensile and compressive forces on the steerable liner 406 through the tension device 402 and the rotational steering device 404.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

Those skilled in the art will appreciate that variations from the specific embodiments disclosed above are contemplated by the invention. The following invention should not be restricted to the above embodiments, but should be measured by the following claims.

What is claimed is:

1. A steerable catheter device comprising:
an elongated catheter body defining a proximal portion and a distal portion and a lumen along the elongated catheter body, the lumen having a proximal end and a distal end with a reinforcing coil disposed within the lumen for supporting the elongated catheter body, the lumen containing a steerable wire extending proximate or within the lumen from the proximal end of the lumen to a distal tip defined at the distal end of the elongated catheter body, wherein the steerable wire is affixed to the distal tip, and wherein actuation of the steerable wire at the proximal end of the elongated catheter body causes at least one of a rotation and a deflection of the distal tip.

2. The device of claim 1 further comprising:
a structural defect located at the distal end of the elongated catheter body, wherein the structural defect aids in the rotation and the deflection of the distal tip.

3. The device of claim 2 wherein the distal tip deflects between zero to ninety degrees.

4. The device of claim 3 wherein the distal tip deflects between twenty to forty-five degrees.

5. The device of claim 1 wherein the elongated catheter body has a diameter of less than 1.5 mm.

6. The device of claim 1 wherein the elongated catheter body has a diameter between 1 mm to 5 mm.

7. The devise of claim 1 wherein the distal tip has generally hemispherical configuration.

8. The devise of claim 1 wherein the distal tip further comprises a plurality of holes.

9. The device of claim 1 further comprising:
a steering mechanism directly engaged with the steerable wire, the steering mechanism to actuate the steerable wire to rotate and deflect the distal tip.

10. The device of claim 9 wherein the steering mechanism further comprises a locking mechanism mechanically engaged with the steerable wire to maintain a constant tension on the steerable wire.

11. The device of claim 1 wherein actuation of the steerable wire at the proximal end of the elongated catheter body causes the distal tip to deflect from a longitudinal axis of the catheter.

12. The device of claim 1 wherein actuation of the steerable wire at the proximal end of the elongated catheter body causes the distal tip rotate about a longitudinal axis of the catheter.

13. A steerable catheter for delivering a drug to an epidural cavity comprising:
an elongated catheter body defining a proximal portion and a distal portion and a lumen along the elongated catheter body, the lumen having a proximal end and a distal end with a reinforcing coil disposed within the lumen for supporting the elongated catheter body, the lumen containing a steerable liner extending proximate or within the lumen from the proximal end of the lumen to a distal tip defined at the distal end of the elongated catheter body, wherein the steerable liner is affixed to the distal tip, and wherein actuation of the steerable liner at the proximal end of the elongated catheter body causes at least one of a rotation and a deflection of the distal tip.

14. The device of claim 13 further comprising:
a structural defect at the distal end of the elongated catheter body, wherein the structural defect aids in the rotation and the deflection of the distal tip.

15. The device of claim 14 wherein the distal tip deflects between zero to ninety degrees.

16. The device of claim 15 wherein the distal tip deflects between twenty to forty-five degrees.

17. The device of claim 13 wherein the elongated catheter body has a diameter of less than 1.5 mm.

18. The device of claim 13 wherein the elongated catheter body has a diameter between 1 mm to 5 mm.

19. The devise of claim 13 wherein the distal tip has a generally hemispherical configuration.

20. The devise of claim 13 wherein the distal tip further comprises a plurality of holes.

21. The device of claim 13 further comprising:
a steering mechanism directly engaged with the steerable liner, the steering mechanism to actuate the steerable liner to rotate and deflect the distal tip.

22. The device of claim 21 wherein the steering mechanism further comprises a locking mechanism mechanically engaged with the steerable liner to maintain a constant tension on the steerable liner.

23. The device of claim 13 wherein the steerable liner has a generally tubular configuration.

24. The device of claim 13 wherein the steerable liner has a solid surface.

25. The device of claim 13 wherein the steerable liner has a generally tubular configuration.

26. The device of claim 13 wherein actuation of the steerable liner at the proximal end of the elongated catheter body causes the distal tip to deflect from a longitudinal axis of the catheter.

27. The device of claim 13 wherein actuation of the steerable liner at the proximal end of the elongated catheter body causes the distal tip to deflect from a longitudinal axis of the catheter.

28. The device of claim 13 wherein actuation of the steerable liner at the proximal end of the elongated catheter body causes the distal tip rotate about a longitudinal axis of the catheter.

29. A method for delivering a drug to an epidural space comprising:
accessing an area adjacent to the epidural space using a steerable catheter comprising:
an elongated catheter body defining a proximal portion and a distal portion and a lumen along the elongated catheter body, the lumen having a proximal end and a distal end with a reinforcing coil disposed within the lumen for supporting the elongated catheter body, the lumen containing a steerable wire extending proximate or within the lumen from the proximal end of the lumen to a distal tip defined at the distal end of the elongated catheter body, wherein the steerable wire is affixed to the distal tip, wherein actuation of the steerable wire at the proximal end of the elongated catheter body causes at least one of a rotation and a deflection of the distal tip;
applying a force to the steering mechanism to at least one of a deflection and a rotation of the distal end into the epidural space;
providing a liquid through the lumen of the catheter;
transmitting the liquid from the proximal end of the lumen to the distal end of the lumen positioned within the epidural space; and
removing the elongated catheter body.

30. A method for manufacturing a steerable catheter device comprising:
forming an elongated catheter body defining a distal portion and a proximal portion, the elongated catheter body defining a lumen having a proximal end and a distal end, wherein the distal portion of the elongated catheter body defining a distal tip; and
inserting a steerable wire along the lumen from the proximal portion of the elongated catheter body to the distal tip, wherein actuating the steerable wire causes at least one of a rotation and a deflection of the distal tip; and
affixing an end of the steerable wire to the distal tip.

31. A method for manufacturing a steerable catheter device comprising:
forming an elongated catheter body defining a distal portion and a proximal portion, the elongated catheter body defining a lumen having a proximal end and a distal end, wherein the distal portion of the elongated body defining a distal tip; and
inserting a steerable wire along the lumen from the proximal portion of the elongated catheter body to the distal tip, wherein actuating the steerable wire causes at least one of a rotation and a deflection of the distal tip; and
affixing an end of the steerable line to the distal tip.
